(19) United States
(12) Patent Application Publication
(10) Pub. No.: US 2002/0052312 A1
(43) Pub. Date: May 2, 2002

(54) Combination therapy of chronic obstructive pulmonary disease using muscarinic receptor antagonists

(76) Inventors: Theodore F. Reiss, Summit, NJ (US); Mark A. Bach, Scotch Plains, NJ (US); Sui-Long Yao, West Windsor, NJ (US)

Correspondence Address: MERCK AND CO INC, P O BOX 2000, RAHWAY, NJ, 070650907

(21) Appl. No.: 09/867,142

(22) Filed: May 29, 2001

Related U.S. Application Data
(60) Provisional application No. 60/207,923, filed on May 30, 2000.

(51) Int. Cl. A01N 37/18; A61K 38/00; A61K 31/57; A61K 31/522

(52) U.S. Cl. 514/2; 514/171; 514/261

(57) ABSTRACT

The present invention provides a method for the treatment of chronic obstructive pulmonary disease using an oral muscarinic receptor antagonists in combination with at least one other therapeutic agent, as well as combination dosage forms therefor.

### BACKGROUND OF THE INVENTION

Chronic obstructive pulmonary disease (COPD) is persistent obstruction of the airways caused by emphysema or inflammation of the small airways in chronic bronchitis. In the United States, about 14 million people suffer from chronic obstructive pulmonary disease. It's second only to heart disease as a cause of disability that makes people stop working, and it's the fourth most common cause of death.

There is currently no ideal therapy for COPD. Although oxygen therapy has demonstrated a survival benefit in severe COPD patients and only smoking cessation has been shown to slow the accelerated decline in respiratory function, the foundation of therapy for patients with COPD is the use of anticholinergic agents such as the quaternary ammonium compounds ipratropium bromide, oxitropium bromide and tiotropium bromide. The mode of action of anticholinergic drugs is not clearly understood; they may act by inhibiting normal, cholinergically mediated bronchomotor tone.

β₂-Agonists, such as metaproterenol, albuterol, terbutaline and pirbuterol, are also used in the treatment of COPD, although generally less effective than the anticholinergics. Both anticholinergics and β2-agonists are administered via inhalation requiring multiple doses per day. The population of COPD patients is generally older and may have less tolerance for sympathomimetic-induced tremor, nervousness, and cardiac side effects of the β₂-agonists than asthmatic patients. However, the use of both an anticholinergic agent and a β₂-agonist in COPD may result in a synergistic response, with increased FEV₁ and FVC values than either agent alone. The combination product Combivent® (Boehringer Ingelheim) is an inhalation product containing ipratropium bromide and albuterol.

Though oral and inhaled steroids are frequently used to treat COPD patients, results of large scale clinical trials have shown poor efficacy, with less than 20-30% of patients achieving any benefit from chronic corticosteroid therapy.

Theophylline's potential for toxicity has led to a decline in its clinical use, but it still retains an important role in COPD treatment. It is of particular value for less compliant or less capable patients who have trouble using aerosol therapy optimally and those patients troubled by nocturnal symptoms. Toxicity can be minimized by combining a low dose of theophylline with an inhaled β2-agonist; bronchodilation is additive, not synergistic.

The quaternary ammonium compounds such as ipratropium bromide and tiotropium bromide are non-selective muscarinic receptor antagonists. There are five known subtypes of muscarinic receptor, M₁-M₅. M₃ mediated stimulation of smooth muscle can result in diverse effects which include bronchoconstriction, visual accommodation, gastrointestinal peristalsis, salivation, and increased detrusor muscle tone in the urinary bladder. M₁, M₂, and M₃ muscarinic receptors have been found throughout the human lung although the role of M₁ receptors, which are predominantly found in alveolar walls, is not well elucidated. M₂ receptors are inhibitory in function (autoreceptors) and serve to inhibit the release of the acetylcholine to which they respond, whereas, M₃ receptors mediate bronchoconstriction. Previous studies in animals and humans have suggested that inhibition of M₂ cholinoceptors (resulting in augmented acetylcholine release) during M₃ inhibition could paradoxically increase bronchoconstriction by increasing the amount of stimulatory (and, hence, bronchoconstrictive) acetylcholine at the M₃ receptor. Consequently, it is hypothesized that an agent which selectively antagonizes the M₃ receptor but spares the M₂ receptor may more effectively facilitate bronchodilatation and be useful in the treatment of COPD. In addition, an M₃-receptor antagonist could similarly minimize other M₂-mediated side effects such as tachycardia.

There is a continuing need for improved therapy for COPD that would offer enhanced efficacy and better side effect profile than currently available treatment methods.

### SUMMARY OF THE INVENTION

The present invention concerns a method for treating patients with chronic obstructive pulmonary disease using an oral muscarinic antagonist in combination with at least one other therapeutic agent. The invention further provides a pharmaceutical composition containing an oral muscarinic antagonist in combination with at least one other therapeutic agent.

### DETAILED DESCRIPTION OF THE INVENTION

The present invention provides a method for the treatment of chronic obstructive pulmonary disease in a patient in need of such treatment, which comprises administering orally to said patient a therapeutically effective amount of a muscarinic M3 receptor antagonist in combination with a therapeutically effective amount of at least one other therapeutic agent selected from the group consisting of: β2-agonist, antitussive, corticosteroid, decongestant, histamine H1 antagonist (antihistamine), dopamine antagonist, leukotriene antagonist, 5-lipooxygenase inhibitor, phosphodiesterase IV inhibitor, VLA-4 antagonist, and theophylline.

In another aspect the present invention provides a pharmaceutical composition suitable for oral administration comprising a therapeutically effective amount of a muscarinic M3 receptor antagonist in combination with a therapeutically effective amount of at least one other therapeutic agent selected from the group consisting of: β2-agonist, antitussive, corticosteroid, decongestant, histamine H1 antagonist, dopamine antagonist, leukotriene antagonist, 5-lipooxygenase inhibitor, phosphodiesterase IV inhibitor, VLA-4 antagonist, and theophylline, and a pharmaceutically acceptable carrier.

Suitable muscarinic M3 receptor antagonists are preferably those that are selective for the M3 subtype, for example those disclosed in U.S. Pat. No. 5,948,792, U.S. Pat. No. 5,750,540 and EP Published Application 863,141. Preferred muscarinic M3 receptor antagonists are those having the formula I or a pharmaceutically acceptable salt thereof:

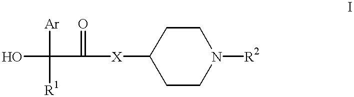

wherein Ar represents an aryl group or a heteroaryl group having 1 to 2 hetero atoms selected from a group consisting of nitrogen, oxygen and sulfur (any 1 to 3 hydrogen atoms on the ring of said aryl or heteroaryl group may be substituted with lower alkyl, trifluoromethyl, cyano, hydroxyl, nitro, lower alkoxycarbonyl, halogen, lower alkoxy, amino or lower alkylamino);

R¹ represents C₃-C₆ cycloalkyl substituted with 1 to 4 fluorine atom(s);

R² represents C₅-C₁₅ saturated or unsaturated aliphatic hydrocarbon groups whose any 1 to 6 hydrogen atoms may be substituted with fluorine atom(s), aralkyl, arylalkenyl, heteroarylalkyl or heteroarylalkenyl group having 1 to 2 hetero atoms selected from a group consisting of nitrogen, oxygen and sulfur (optionally any 1 to 3 hydrogen atoms on the ring in said aralkyl, arylalkenyl, heteroarylalkyl or heteroarylalkenyl group may be substituted with lower alkyl, trifluoromethyl, cyano, hydroxyl, nitro, lower alkoxycarbonyl, halogen, lower alkoxy, amino or lower alkylamino); and

X stands for O or NH, provided that at least either one of R¹ and R² contains one or more fluorine atoms.

More particularly, the M3 muscarinic M3 receptor antagonist is the compound having the formula Ia:

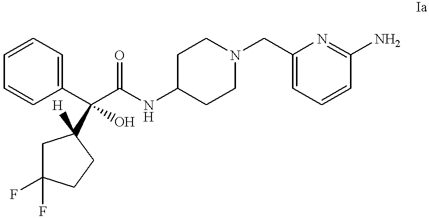

and pharmaceutically acceptable salts thereof.

Example of suitable β2-agonists include albuterol, terbutaline and metaproterenol.

Examples of antitussives include, but are not limited to, dextromethorphan, chlophedianol, carbetapentane, caramiphen, noscapine, diphenhydramine, codeine, hydrocodone, hydromorphone, fominoben, benzonatate, pharmaceutically acceptable salts thereof and mixtures thereof.

Examples of decongestants suitable for use in the present invention include pseudoephedrine, phenylpropanolamine, phenylephrine, ephedrine, pharmaceutically acceptable salts thereof and mixtures thereof.

Examples of leukotriene antagonist include, but are not limited to, montelukast, zafirlukast, pranlukast and pharmaceutically acceptable salts thereof.

Examples of 5-lipooxygenase inhibitors include zileuton, atreluton and the comopound MK-591 (i.e. 3-[N-(p-chlorobenzyl)-3-(t-butylthio)-5-(quinolin-2 -ylmethoxy)indol-2-yl]-2,2-dimethylpropanoic acid).

Examples of antihistamines include, but are not limited to, azelastine, acrivastine, cyclizine, carebastine, cyproheptadine, carbinoxamine, doxylamine, dimethindene, ebastine, epinastine, efletirizine, ketotifen, levocabastine, mizolastine, mequitazine, mianserin, noberastine, meclizine, norastemizole, picumast, tripelenamine, temelastine, trimeprazine, triprolidine, bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine, pyrilamine, astemizole, terfenadine, loratadine, cetirizine, levocetirizine, fexofenadine, descarboethoxyloratadine. Preferred anthistamines include loratadine, fexofenadine, cetirizine, descarboethoxyloratadine, astemizole, noraztemizole, and levocetirizine.

Examples of phosphodiesterase IV inhibitors include Ariflo® (SmithKline Beecham), and roflumilast.

Examples of corticosteroids include prednisone.

VLA-4 antagonists are compounds which block the binding of VLA-4 (very late antigen-4; α4β1) to its ligands. Examples of VLA-4 antagonists are disclosed in U.S. Pat. No. 5,510,332, WO00/18759, WO00/18760, WO00/15612, WO00/05224, WO00/05223, WO00/01690, WO00/00477, WO99/67230, WO99/61465, WO99/54321, WO99/47547, WO99/43642, WO99/37618, WO99/37605, WO99/36393, WO99/35163, WO99/24398, WO99/23063, WO98/58902, WO98/54207, WO97/03094, WO97/02289, WO96/40781, WO96/40641, WO96/31206, WO96/22966, WO96/20216, WO96/06108, WO96/01644, WO95/15973, EP0918059A1, EP0842943A2, EP0905139A2, EP0903353A1, WO98/53818, WO98/53814 and WO98/53817.

The term “therapeutically effective amount” means an amount that produces the desired therapeutic response upon oral administration, and can be readily determined by one skilled in the art.

The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a muscarinic M3 receptor antagonis and another therapeutically active ingredient as enumerated above, and pharmaceutically acceptable excipients.

It is understood that as used herein the therapeutically active ingredients (muscarinic M3 receptor antagonists, β2-agonists, leukotriene antagonists, etc.) encompass pharmaceutically acceptable salts of the active chemical entities. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-dibenzylethylenediamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When a compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

In one embodiment, the present invention provides a pharmaceutical composition suitable for oral administration comprising a therapeutically effective amount of a muscarinic M3 receptor antagonist in combination with a therapeutically effective amount of a β2-agonist, and a pharmaceutically acceptable carrier, and a method for the treatment of COPD by adminsitering said composition to a patient in need of such treatment. In a preferred embodiment, the muscarinic M3 receptor antagonist is a compound of formula I; more preferably, a compound of formula Ia. In another preferred embodiment, the β2-agonist is selected from albuterol, terbutaline and metaproterenol.

In another embodiment, the present invention provides a pharmaceutical composition suitable for oral administration comprising a therapeutically effective amount of a muscarinic M3 receptor antagonist in combination with a therapeutically effective amount of an antitussive, and a pharmaceutically acceptable carrier, and a method for the treatment of COPD by adminsitering said composition to a patient in need of such treatment. In a preferred embodiment, the muscarinic M3 receptor antagonist is a compound of formula I; more preferably, a compound of formula Ia. In another preferred embodiment, the antitussive is selected from the group consisting of dextromethorphan, chlophedianol, carbetapentane, caramiphen, noscapine, diphenhydramine, codeine, hydrocodone, hydromorphone, fominoben, and benzonatate.

In another embodiment, the present invention provides a pharmaceutical composition suitable for oral administration comprising a therapeutically effective amount of a muscarinic M3 receptor antagonist in combination with a therapeutically effective amount of a decongestant, and a pharmaceutically acceptable carrier, and a method for the treatment of COPD by adminsitering said composition to a patient in need of such treatment. In a preferred embodiment, the muscarinic M3 receptor antagonist is a compound of formula I; more preferably, a compound of formula Ia. In another preferred embodiment, the decongestant is selected from the group consisting of pseudoephedrine, phenylpropanolamine, phenylephrine and ephedrine.

In another embodiment, the present invention provides a pharmaceutical composition suitable for oral administration comprising a therapeutically effective amount of a muscarinic M3 receptor antagonist in combination with a therapeutically effective amount of a histamine HI antagonist, and a pharmaceutically acceptable carrier, and a method for the treatment of COPD by adminsitering said composition to a patient in need of such treatment. In a preferred embodiment, the muscarinic M3 receptor antagonist is a compound of formula I; more preferably, a compound of formula Ia. In another preferred embodiment, the histamine Hi antagonist is selected from loratadine, fexofenadine, cetirizine, descarboethoxyloratadine, astemizole, noraztemizole, and levocetirizine; more preferably loratadine, fexofenadine, cetirizine or descarboethoxyloratadine.

In another embodiment, the present invention provides a pharmaceutical composition suitable for oral administration comprising a therapeutically effective amount of a muscarinic M3 receptor antagonist in combination with a therapeutically effective amount of a leukotriene antagonist, and a pharmaceutically acceptable carrier, and a method for the treatment of COPD by adminsitering said composition to a patient in need of such treatment. In a preferred embodiment, the muscarinic M3 receptor antagonist is a compound of formula I; more preferably, a compound of formula Ia. In another preferred embodiment, the leukotriene antagonist is selected from montelukast, pranlukast and zafirlukast; more preferably montelukast, particularly montelukast sodium.

In another embodiment, the present invention provides a pharmaceutical composition suitable for oral administration comprising a therapeutically effective amount of a muscarinic M3 receptor antagonist in combination with a therapeutically effective amount of a 5-lipooxygenase inhibitor, and a pharmaceutically acceptable carrier, and a method for the treatment of COPD by adminsitering said composition to a patient in need of such treatment. In a preferred embodiment, the muscarinic M3 receptor antagonist is a compound of formula I; more preferably, a compound of formula Ia. In another preferred embodiment, the 5-lipooxygenase is zileuton or MK-591 having the formula II or a pharmaceutically acceptable salt thereof:

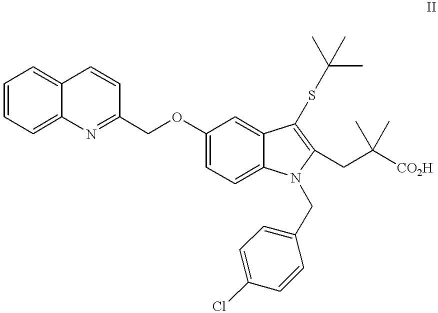

In another embodiment, the present invention provides a pharmaceutical composition suitable for oral administration comprising a therapeutically effective amount of a muscarinic M3 receptor antagonist in combination with a therapeutically effective amount of a phosphodiesterase IV inhibitor, and a pharmaceutically acceptable carrier, and a method for the treatment of COPD by adminsitering said composition to a patient in need of such treatment. In a preferred embodiment, the muscarinic M3 receptor antagonist is a compound of formula I; more preferably, a compound of formula Ia. In another preferred embodiment, the phosphodiesterase IV inhibitor is cilomilast (Ariflo®) or roflumilast.

Dosage and Administration

In the present method, the muscarinic M3 receptor antagonist and the second therapeutic agent may be administered separately in separate dosage forms or together in a single unit dosage form. Where separate dosage formulations are used, the therapeutic agents can be administered at essentially the same time, i.e., concurrently, or at separately staggered times, i.e, sequentially. It is preferred that the therapeutic agents be co-administered concurrently on a once-a-day dosing schedule; however, varying dosing schedules, such as one of the therapeutic agent used once per day and the other therapeutic agent once, twice or more times per day, is also encompassed herein. A single oral dosage formulation comprised of both therapeutic agents is preferred. A single dosage formulation will provide convenience for the patient.

Each of the therapeutic agent may be administered at a dosage level up to the conventional dosage levels used for monotherapy. Suitable dosage levels will depend upon the chosen muscarinic M3 receptor antagonist and the second therapeutic agent, but typically suitable levels for each therapeutic agent will be about 0.001 to 50 mg/kg body weight of the patient per day, preferably 0.005 to 30 mg/kg per day, and especially 0.05 to 10 mg/kg per day. The compounds may be administered on a regimen of up to 6 times per day, preferably 1 to 4 times per day, and especially once per day. The actual dosage employed may be varied depending upon the particular therapeutically active ingredient chose, and the patient's age, sex, weight, and severity of the condition being treated. The selection of the appropriate dosage, including amount and frequency, may be readily practiced by a physician skilled in the art.

Pharmaceutical Compositions

The instant invention also provides pharmaceutical compositions comprised of a therapeutically effective amount of a muscarinic M3 receptor antagonist in combination with a therapeutically effective amount of another therapeutic agent as enumerated above, and a pharmaceutically acceptable carrier. One embodiment of the instant compositions is a single composition adapted for oral administration. Pharmaceutical compositions of the present invention suitable for oral administration may be presented as discrete units such as hard or soft gelatin capsules (solid-filled, semi-solid filled, or liquid filled), cachets or tablets each containing a predetermined amount of the active ingredient, as a powder for reconstitution or dispersible granules, as oral gels, elixirs, syrups, or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredients with the carrier which constitutes one or more necessary ingredients.

In general, the compositions are prepared by uniformly and intimately admixing the active ingredients with liquid carriers or finely divided solid carriers or both, and then, if necessary, shaping the product into the desired presentation. Commonly used carriers may be, for example, corn starch, talc, calcium phosphate, calcium sulphate, calcium stearate, magnesium stearate, steane acid, sorbitol, microcrystalline cellulose, mannitol, gelatin, natural or synthetic gums, such as carboxymethylcellulose, methylcellulose, alginate, dextran, acacia gum, karaya gum, locust bean gum. Additionally, other excipients such as diluents, binders, lubricants, disintegrants, colors and flavoring agents may be employed. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. The dosage form can also be film coated. Desirably, each tablet contains from about 1 mg to about 500 mg of each of the active ingredient and each cachet or capsule contains from about 1 to about 500 mg of each of the active ingredient.

In addition to the common dosage forms set out above, the therapeutically active ingredients may also be administered by controlled release means and/or delivery devices to provide the rate-controlled release of any one or more of the components or active ingredients to optimize the desired therapeutic effects. Suitable dosage forms for sustained release include layered tablets containing layers of varying disintegration rates or controlled release polymeric matrices inpregnated with the active components and shaped in tablet form or capsules containing such impregnated or encapsulated porous polymeric matrices. Examples of such controlled release means are described in U.S. Pat. Nos. 3,845,770; 3,916,899; 3,536,809; 3,598,123; 3,630,200 and 4,008,719.

### EXAMPLE 1

### Combination Tablet Preparation

Tablets containing 25.0, 50.0, and 100.0 mg, respectively, of a muscarinic M3 antagonist of formula I and 10 mg of montelukast sodium are prepared as illustrated below:

| | Amount-mg | | |
|---|---|---|---|
| M3 antagonist of formula I | 25.0 | 50.0 | 100.0 |
| montelukast sodium | 10.0 | 10.0 | 10.0 |
| Microcrystalline cellulose | 37.25 | 100.0 | 175.0 |
| Modified food corn starch | 37.25 | 4.25 | 8.5 |
| Magnesium stearate | 0.50 | 0.75 | 1.5 |

Both active compounds, cellulose, and a portion of the corn starch are mixed and granulated to 10% corn starch paste. The resulting granulation is sieved, dried and blended with the remainder of the corn starch and the magnesium stearate. The resulting granulation is then compressed into tablets containing 25.0, 50.0, and 100.0 mg, respectively, of muscarinc M3 antagonist per tablet, and 10 mg of montelukast sodium per tablet.

What is claimed is:

1. A method for the treatment of chronic obstructive pulmonary disease in a patient in need of such treatment, which comprises administering orally to said patient a therapeutically effective amount of a muscarinic M3 receptor antagonist in combination with a therapeutically effective amount of at least one other therapeutic agent selected from the group consisting of: β2-agonist, antitussive, corticosteroid, decongestant, histamine H1 antagonist (antihistamine), dopamine antagonist, leukotriene antagonist, 5-lipooxygenase inhibitor, phosphodiesterase IV inhibitor, VLA-4 antagonist, and theophylline.

2. A method of claim 1 wherein said other therapeutic agent is a β2-agonist.

3. A method of claim 1 wherein said other therapeutic agent is a antitussive.

4. A method of claim 1 wherein said other therapeutic agent is a corticosteroid.

5. A method of claim 1 wherein said other therapeutic agent is a decongestant.

6. A method of claim 1 wherein said other therapeutic agent is a histamine H1 antagonist.

7. A method of claim 1 wherein said other therapeutic agent is a leukotriene antagonist.

8. A method of claim 1 wherein said other therapeutic agent is a 5-lipooxygenase inhibitor.

9. A method of claim 1 wherein said other therapeutic agent is a phosphodiesterase IV inhibitor.

10. A method of claim 1 wherein said other therapeutic agent is theophylline.

11. A pharmacuetical composition suitable for oral administration comprising a therapeutically effective amount of a muscarinic M3 receptor antagonist in combination with a therapeutically effective amount of at least one other therapeutic agent selected from the group consisting of: β2-agonist, antitussive, corticosteroid, decongestant, histamine H1 antagonist, dopamine antagonist, leukotriene antagonist, 5-lipooxygenase inhibitor, phosphodiesterase IV inhibitor, VLA-4 antagonist, and theophylline, and a pharmaceutically acceptable carrier.

12. A pharmaceutical composition of claim 11 wherein said other therapeutic agent is a β2-agonist.

13. A pharmaceutical composition of claim 11 wherein said other therapeutic agent is a antitussive.

14. A pharmaceutical composition of claim 11 wherein said other therapeutic agent is a corticosteroid.

15. A pharmaceutical composition of claim 11 wherein said other therapeutic agent is a decongestant.

16. A pharmaceutical composition of claim 11 wherein said other therapeutic agent is a histamine HI antagonist.

17. A pharmaceutical composition of claim 11 wherein said other therapeutic agent is a leukotriene antagonist.

24. A pharmaceutical composition of claim 11 wherein said other therapeutic agent is a 5-lipooxygenase inhibitor.

18. A pharmaceutical composition of claim 11 wherein said other therapeutic agent is a phosphodiesterase IV inhibitor.

19. A pharmaceutical composition of claim 11 wherein said other therapeutic agent is theophylline.
